(19) United States
(12) Patent Application Publication
(10) Pub. No.: US 2013/0150711 A1
(43) Pub. Date: Jun. 13, 2013

(54) NON-INVASIVE MEDICAL CONDITION MONITORING APPARATUS

(71) Applicant: PneumoSonics, Inc., Cleveland, OH (US)

(72) Inventors: David M. THEOBOLD, Chapel Hill, NC (US); David Arthur BLUM, Boston, MA (US); Gregory T. SCHULTE, Minneapolis, MN (US); Alan GRESZLER, Westlake, OH (US); Vincent OWENS, Hingham, MA (US)

(73) Assignee: PneumoSonics, Inc., Cleveland, OH (US)

(21) Appl. No.: 13/708,276

(22) Filed: Dec. 7, 2012

Related U.S. Application Data
(60) Provisional application No. 61/569,069, filed on Dec. 9, 2011.

(51) Int. Cl. A61B 5/00

(52) U.S. Cl. CPC A61B 5/68; A61B 5/6852
USPC 600/424; 600/407

(57) ABSTRACT

Embodiments of the present invention may provide a detector device including a communication interface to couple the detection device to an external antenna device. The detector device may detect medical condition(s) by performing a reference scan and a subsequent target scan. The target scan may be compared to the reference scan, and deviations from the reference scan may indicate the presence of the medical condition(s). According to embodiments of the present invention, the detection device may continuously monitor for medical condition(s) by performing an initial reference scan and subsequent target scans. Each target scan may be compared to the reference scan, and deviations from the reference scan may indicate the presence of the medical condition(s) or a change in the medical condition(s).

### RELATED APPLICATIONS

This application claims priority to U.S. Patent Application Ser. No. 61/569,069 filed on Dec. 9, 2011, the content of which is incorporated herein in its entirety.

### BACKGROUND

The present invention relates to detector devices for monitoring medical conditions using micropower impulse radar (MIR) technology.

Medical conditions often present themselves as a change in body composition. For example, a pneumothorax is a medical condition where a pocket of air is trapped in the pleural space around the lungs, making breathing difficult. In some cases, pneumothorax can lead to a collapse of a lung and possibly even death. It is most often caused by blunt trauma to the chest, such as the trauma experienced in some car accidents.

Pneumothorax can also be caused by errors in medical procedures such as central line placement. Typically, after a central line placement, the patient receives an x-ray or ultrasound to detect for a possible pneumothorax. However, pneumothorax diagnosis by x-rays or ultrasounds is cumbersome. For example, x-ray or ultrasound imaging systems are generally not portable and, thus, the patient has to be brought to the equipment. Also, a skilled professional (i.e., a doctor) must usually interpret the x-ray or ultrasound images for pneumothorax diagnosis. Moreover, x-rays or ultrasounds are not suitable for continuous monitoring of a pneumothorax during or after a medical procedure.

### BRIEF DESCRIPTION OF THE DRAWINGS

FIG. 1 is a simplified block diagram of a medical condition monitoring system according to an embodiment of the present invention.

FIG. 2 illustrates a data flow diagram of scan processing operations of a detector device according to an embodiment of the present invention.

FIG. 3 illustrates a data flow diagram of a scan processing technique using envelope detection with a synthetic quadrature channel according to an embodiment of the present invention.

FIG. 4(a) is a graph of data generated by scan processing operations of a detector device according to an embodiment of the present invention.

FIG. 4(b) is a normalized graph of the data samples of FIG. 6(a)

FIG. 4(c) is a graph of low-pass filtered results of data generated by scan processing operations of a detector device according to an embodiment of the present invention.

FIG. 4(d) is a graph of a synthetic quadrature channeled formed by performing a derivative function on data generated by scan processing operations of a detector device according to an embodiment of the present invention.

FIG. 4(e) is a normalized graph of the data samples of FIG. 4(d)

FIG. 4(f) is a graph of envelope detection performed on data generated by scan processing operations of a detector device according to an embodiment of the present invention.

FIG. 4(g) is a graph of low-pass filtered results of the data samples of FIG. 4(f).

FIG. 5 illustrates a feedback loop using systems of the present invention to control therapy delivery in a medical device according to an embodiment of the present invention.

FIG. 6 is a simplified block diagram of an integrated MIR/stimulation system according to an embodiment of the present invention.

FIG. 7 illustrates a flow diagram of an MIR scanning system integrated with an alert system according to an embodiment of the present invention.

FIG. 8 is a simplified block diagram of an integrated MIR/alert system according to an embodiment of the present invention.

### DETAILED DESCRIPTION

Embodiments of the present invention may provide a detector device including a communication interface to couple the detection device to an external antenna device. The detector device may detect medical condition(s) by performing a reference scan and a subsequent target scan. The target scan may be compared to the reference scan, and deviations from the reference scan may indicate the presence of the medical condition(s).

According to embodiments of the present invention, the detection device may continuously monitor for medical condition(s) by performing an initial reference scan and subsequent target scans. Each target scan may be compared to the reference scan, and deviations from the reference scan may indicate the presence of the medical condition(s) or a change in the medical condition(s).

The detection device may be used in a non-invasive medical condition monitoring system for patients using MIR technology. The medical condition can be a medical disorder, dysfunction or other abnormality. The patients can be humans or other mammalian subjects. An exemplary system may include a detector and a detachable antenna. The detector may perform a scan by generating one or more MIR pulses that are transmitted into the patient through the antenna, which may be affixed to a specified location on the patient. Reflections or echoes of the pulses from the patient (e.g., muscle, tissue, fluid) may then be captured by the antenna. Electrical signals generated by the antenna device or devices may be interpreted by a processor to detect the presence, location, extent, and volume of a medical disorder, dysfunction or other abnormality.

For example, reflection magnitude (i.e., amount of reflections) and timing deviations may indicate the presence of a medical condition. Conversely, a lack of magnitude and timing deviations may indicate an absence of a medical condition. The medical condition may change the patient's body composition and, consequently, its reflective properties. Different body compositions have different associated impedances. In addition to a change in impedance, the medical condition may change the distance relationships of the body composition, which corresponds to a change in the reflection propagation distance. Therefore, by analyzing the reflection deviation profile, the presence of a medical condition, such as a pneumothorax, for example, may be detected as well as providing an estimation of its approximate volume and depth.

The systems of the present invention may be used to detect or monitor various medical conditions including confirming whether medical treatment results in a therapeutic benefit. Exemplary monitoring and diagnostic uses of the systems of the present invention include detecting and monitoring pneumothoraces (including iatrogenic and traumatic pneumothoraces), hematomas, perforated bowels, fluid pooling in and around tissues such as pericardial effusion and pleural effusion, stomach content changes or distention, changes in bone growth, respiratory function during anesthesia delivery, tumor progression, hemorrhages or aneurysms, and onset of kidney or gallstones.

The systems may also be incorporated with other systems and devices to provide integrated diagnostic or monitoring systems. Exemplary devices include implantable or insertable medical devices, including intravascular devices. A non-limiting example of an implantable device includes an electrical stimulation device and a non-limiting example of an intravascular device includes a catheter. The systems of the present invention may also be integrated with medical intervention monitoring systems.

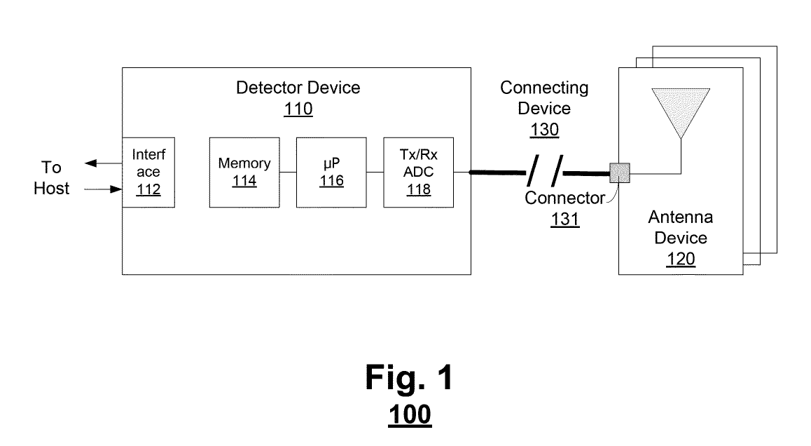

FIG. 1 is a simplified block diagram of a medical condition monitoring system 100 in which embodiments of the present invention may be provided. The system 100 may include a detector device 110, a connecting device 130, and an antenna device 120. The detector device 110 may be coupled to the antenna device 120 through a connecting device 130 via a connector 131.

The detector device 110 may include an interface 112, a memory 114, a processor 116, and transceiver (TX/RX) circuitry 118. The interface 112 may couple the detector device 110 to a remote host system such as a laptop, notebook, tablet computer, desktop computer or the like. In an embodiment, the interface 112 may be a USB port. In another embodiment, the interface 112 may facilitate wireless communication with the host system such as by long range communication (e.g., cellular), short range communication (e.g., WIFI, Bluetooth) or a combination thereof.

The memory 114 may be provided as a volatile memory, a non-volatile memory, or a combination thereof. The memory 114 may store program instructions for the processor 116, scan data generated by the system 100 and any pattern data (discussed below) as needed by the system 100.

The processor 116 may be a microcontroller or a microprocessor. The processor 116 may execute the instructions stored in the memory 116 and may control the operations of the detector device 110.

The TX/RX circuitry 118 may generate MIR pulse(s) and send the pulse(s) to the antenna device 120 to be transmitted as electromagnetic waves into the patient's body. The TX/RX circuitry 118 may also receive corresponding reflections of the transmitted electromagnetic waves captured by the antenna device 120. The components and operations of the TX/RX circuitry 118 may be provided as described in U.S. patent application Ser. No. 12/713,616 filed on Feb. 26, 2010 (published as US 2010/0222663), which is incorporated herein in its entirety.

The connecting device 130 may couple the detector device 110 to the antenna device 120 via the connector 131. In an embodiment, the connecting device 130 may be provided as a coaxial cable. In another embodiment, the connecting device 130 may be provided as a wireless communication network such as WIFI, Bluetooth or the like.

The antenna device 120 may be provided as a planar ultra-wideband antenna and may be detachable from the detector device 110. Responsive to MIR pulse(s) generated by the detector device 110, the antenna device 120 may transmit electromagnetic waves corresponding to the MIR pulse(s). The antenna device 110 may also capture corresponding reflections of the transmitted electromagnetic waves from the patient's body. In an embodiment, the system 100 may include a plurality of antenna devices 120 and connecting devices 130 to provide a multiple antenna array.

In operation, the detector device 110 may generate MIR pulse(s), which cause the antenna device 120 to resonate at its resonant frequency producing electromagnetic radiation. The antenna device 120 may be held in place (e.g., placed against the patient's chest) using an adhesive with little or no air interposing between the patient and antenna. Thus, the electromagnetic radiation may penetrate the patient's body and may be reflected back by various body composition materials. The reflections may be captured by the antenna device 120 and may be processed by the transmitting/receiving circuitry 118 in the detector device 110. After analog baseband processing, the reflections may be digitized. The analog processing including analog-to-digital conversion may be performed as described in U.S. patent application Ser. No. 12/713,616 filed on Feb. 26, 2010 (published as US 2010/0222663).

The digitized reflections may be processed to determine the presence of known medical conditions. The processing may be performed according to stored instructions (i.e., software program(s)) executed by the detector device 110, the coupled host system, or a combination thereof. For example, the detector device 110 may perform a portion of the processing and the host system may perform the remaining processing.

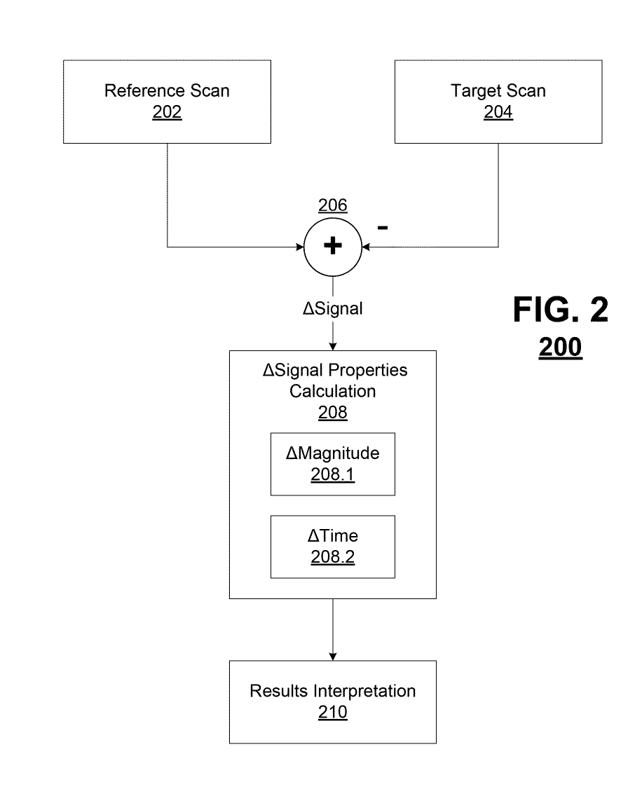

FIG. 2 illustrates is a data flow diagram illustrating scan processing operations of a detector device according to an embodiment of the present invention.

In step 202, a reference scan may be performed. The reference scan may provide a baseline for normal conditions associated with the patient. The reference scan 202 may include driving another activation pulse to the antenna, capturing electromagnetic reflections received by the antenna following the pulse, digitizing the captured EM signals, preprocessing the digitized data, and storing the digitized data for later reference. The reference scan data may be saved in memory for later use, for example, at the detector device, a host system or both. Also, other patient information such as patient ID, any configuration data read from the antenna, location of antenna, etc. may also be saved associated with the reference scan data.

In step 204, a target scan may be performed. The target scan 204 may include driving an activation pulse to the antenna, capturing electromagnetic reflections received by the antenna following the pulse, digitizing the captured EM signals, preprocessing the digitized data, and storing the digitized data for later reference. The target scan may be performed at any time (and, indeed, multiple times) after the reference scan.

In step 206, target scan data may be compared to reference scan data producing a difference signal represented by ΔSignal. The difference signal may indicate patient changes between the target scan and the reference scan.

In step 208, ΔSignal properties may be calculated generating data results. ΔSignal properties may include magnitude and time (Steps 208.1 and 208.2). The ΔSignal properties may indicate the presence or absence of a medical condition as well as estimate the medical condition's characteristics. For example, in a pneumothorax monitoring scenario, the magnitude may indicate the size of the air gap because the magnitude may be proportional to the volume of the air gap. Further, the time may indicate the depth/location of the air gap because the time of a deviation (e.g., sample number) may be proportional to the depth of air gap.

In step 210 the results may be interpreted. For example, the results may be compared to stored profiles of known medical conditions, and if the results match a certain profile, it may indicate the presence of the corresponding medical condition. In an embodiment, the results may be translated into a graphical display element. The graphical display element may convey the changes in the patient's condition as measured by the target scan(s) to a user/technician. For example, the graphical display element may be an icon that changes size and/or color based on the results. The display may be provided on the detector device, the host system or both.

In a medical procedure monitoring scenario, the reference scan may be performed at a time where the patient is known to be free of the medical condition(s) to be detected, typically before a medical procedure is performed on the patient. In some circumstances where the medical condition arises gradually after a procedure, it may be appropriate to perform a reference scan either during performance of the procedure or shortly after it concludes. Regardless of circumstance, the reference scan is taken at a time where the reference scan can be used as a reliable baseline for later scans.

If the medical procedure includes an insertion of a foreign object such as a central venous catheter (CVC) line, the antenna may be placed away from the object so as to not interfere with the scan. In another embodiment where medical conditions at or near the placement of the foreign object require monitoring, the reference scan may be performed immediately after the object insertion. Thus, the reference scan may capture characteristics of the patient's body with the object introduced but before the detected medical condition arises. Also, in an embodiment, the foreign object may not be detectable in the scans. For example, a small plastic catheter may not interfere with the MIR scans, and, therefore, no adjustments for the foreign object may be needed.

According to an embodiment, the target scan is performed with the antenna mounted at the same location on the patient's body as the reference scan. If the antenna is not adhered to the patient properly (e.g., an air gap forms), the improper connection may be detected and an alert to the user may be generated. In an embodiment, if the antenna is moved to another location or exchanged with another antenna between scans, the target scan may still be processed. In an embodiment, antenna configuration changes may be accounted for in the target scan processing.

According to an embodiment, the same connecting device (e.g., cable) between the antenna device and detector device is used in the target scan as the reference scan. However, if the cable is exchanged for another with different properties (e.g., different lengths) between scans, the change may be detected and an alert to the user may be generated. In an embodiment, the cable exchange may be adjusted for in the target scan processing.

In an embodiment, a second antenna may be used on the opposite, unaffected side of the patient to provide a baseline reference. The antennas may be matched or calibrated to be similar in response.

Various signal processing techniques may be utilized for data processing. For example convolution or cross-correlation processing may be implemented to calculate deviation values of the target scan(s) from the reference scan. Convolution processing may be implemented where a target profile (e.g., reference scan profile) is correlated against an observed target signal (e.g., target scan). For example, a tissue/air boundary may be characterized by an abrupt change in dielectric constant, which in turn may cause a step in a detected signal envelope. Thus, convolution processing with a step function may reveal the position and magnitude of such a discontinuity. In an embodiment, envelope detection may be used to calculate deviation values of the target scan(s) from the reference scan. In an embodiment, correlation between a windowed reference scan and windowed test scan may reveal point by point differences between the two scans. Subsequent processing by variance analysis, threshold detection, or similar technique may reveal differences between the two scans.

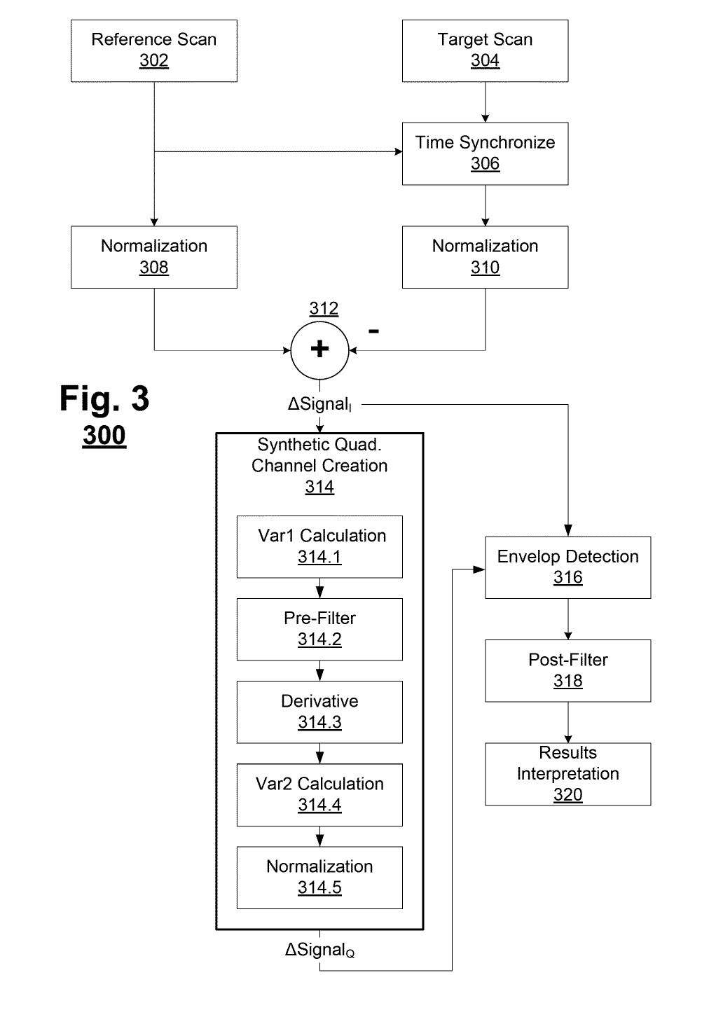

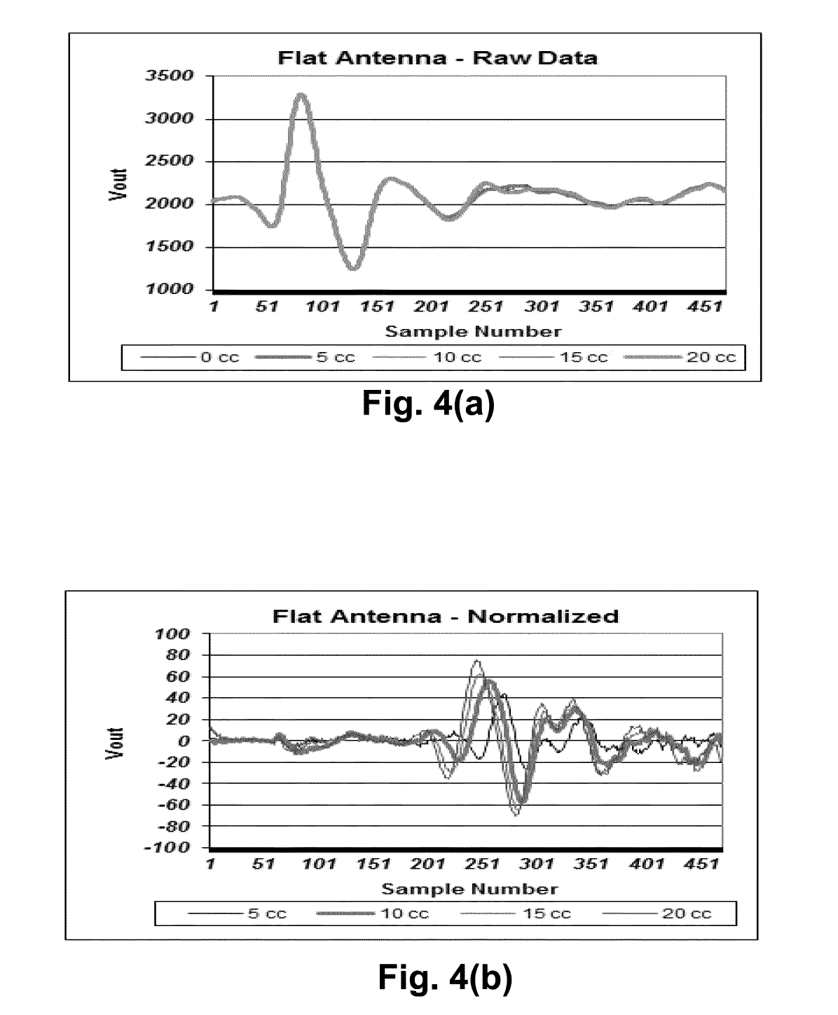

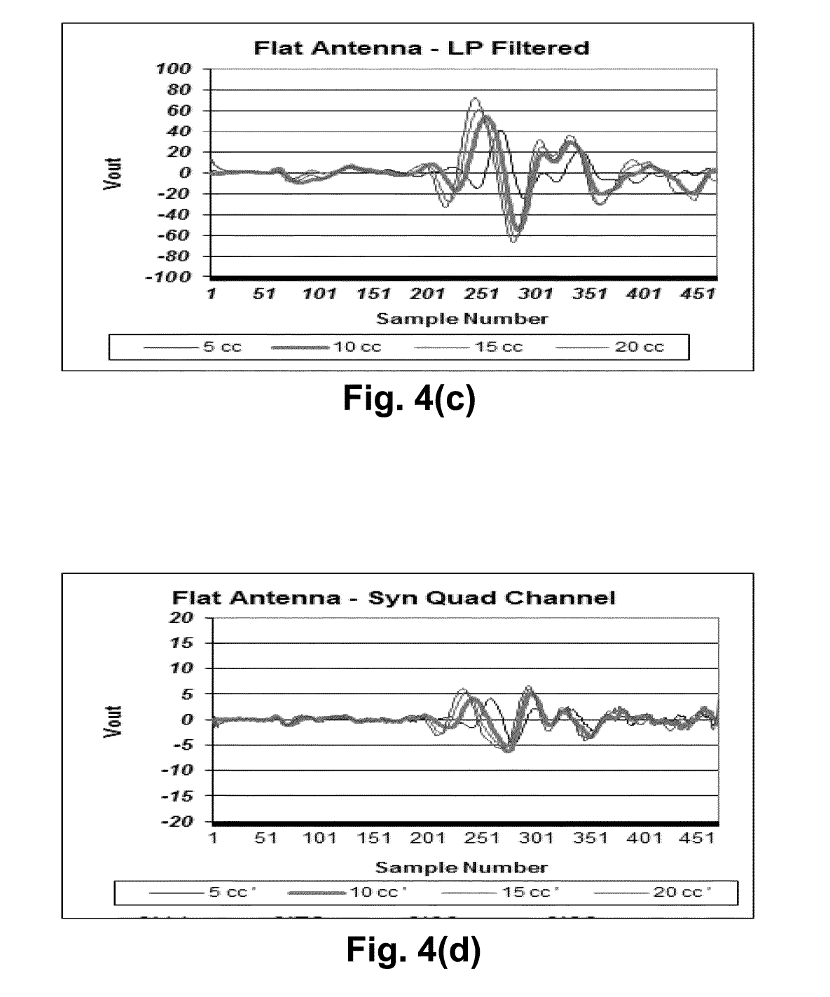

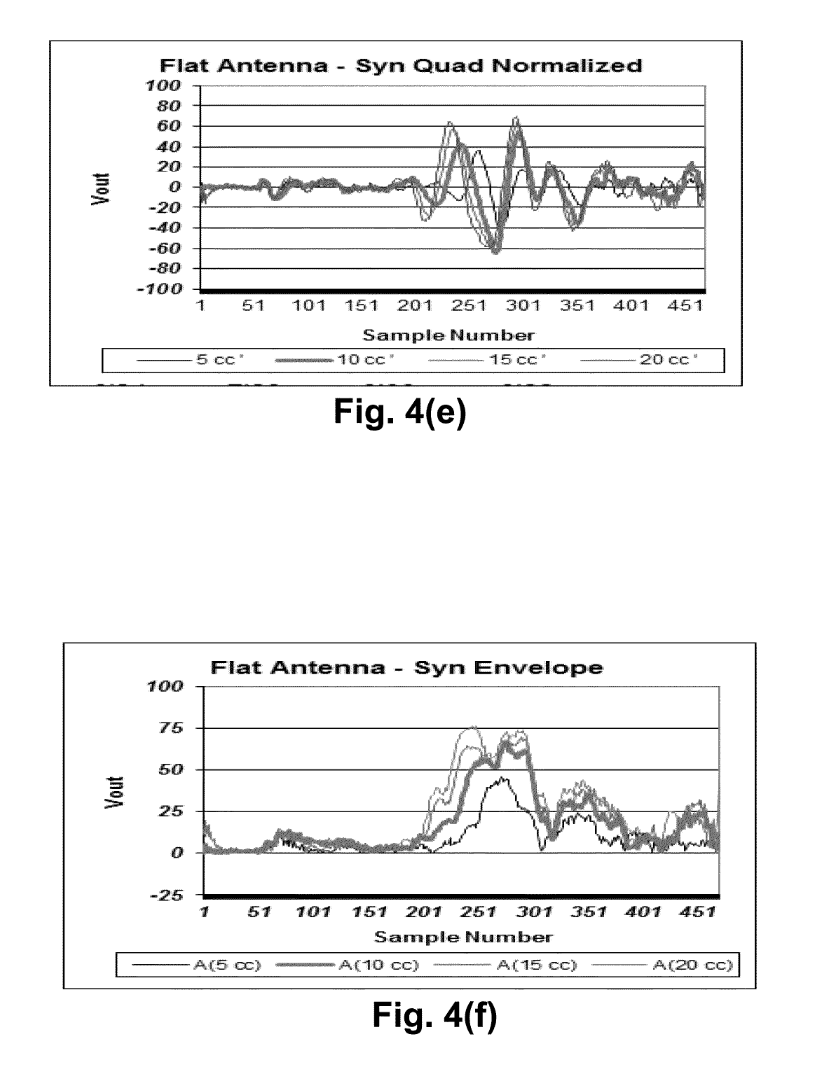

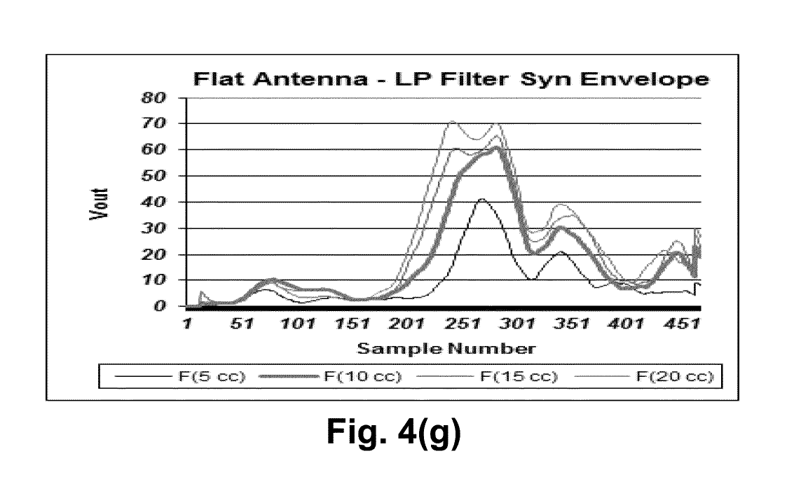

FIG. 3 illustrates a scan processing technique using envelope detection with a synthetic quadrature channel according to an embodiment of the present invention. In steps 302 and 304, a reference scan and a subsequent target scan may be performed similar to steps 202 and 204 described above in the discussion of FIG. 2. For illustration purposes, FIG. 4(a) shows plots of test raw data scans. The plots may represent digital samples from the analog-to-digital conversion.

In step 306, if the target scan shows a time offset from the reference scan, the target scan may be synchronized with the reference scan. Each digitized scan may have a number of samples, and the system may search for peaks in the scans to match them up. In an embodiment, the main pulse of the scans may be used for the synchronization because typically the main pulse has a high amplitude, is relatively noise free, and is relatively stable. The main pulse may correspond to the pulse path in the antenna device. The pathway up to this point may be comprised of well defined materials with known matched impedance and known dielectric properties. Therefore, it may be reliably used as a synchronization reference. In the FIG. 4(a) example, the main pulse may correspond to the points between sample numbers ˜50 and ˜150. In an embodiment, time synchronization may be performed by (1) finding a peak sample in the wave form, “PEAK” (sample number 51 in FIG. 4(a) example), (2) cross-correlating the reference and target samples from points 1 through to the calculated peak, (3) calculating an offset based on the correlation of the peaks, and (3) time shifting the target samples by the offset.

In steps 308 and 310, if the reference and target scans include any biasing errors, the reference samples and target samples may be normalized. Normalization may remove any biasing error from the analog-to-digital conversion by shifting the samples to a bipolar pattern (i.e., the data exhibits the same positive and negative excursions about zero). FIG. 4(b) shows normalized data samples of FIG. 4(a), which were biased at a non-zero value (approximately 2100 Vout).

In step 312, target scan data may be compared to reference scan data producing a difference signal represented by ΔSignal_{I}. In an embodiment, the scans may use a baseband pulse with baseband reflections and the difference signal may only include an in-phase channel ΔSignal_{I}.

In step 314, a synthetic quadrature channel ΔSignal_{Q} may be created for better edge detections. Steps 314.1-314.5 describe a synthetic quadrature channel creation technique according to an embodiment of the present invention; however, other suitable synthetic quadrature channel creation techniques via software or hardware implementation may be used.

In step 314.1, a variance, Var1, of the data may be calculated from the main pulse (i.e., PEAK) through to the end of the data for later scaling purposes if needed. The variance may correspond to the distribution of the data samples from the PEAK. In step 314.2, if the data displays noisy characteristics, the data may be low pass filtered. In an embodiment, the filter may be implemented by a boxcar filter, a multiple coefficient multiplier filter, or other known suitable filters.

In an embodiment, a boxcar filter in the form of an N point (ex. N=9) running average may be used. The filtering may be implemented by (1) maintaining (i.e., not changing) the first four points, (2) for each subsequent point, summing the previous four, the current, and the next four points of the original data, and (3) dividing by nine. FIG. 4(c) shows low pass filtered results from the above-described filtering process.

In step 314.3, a derivative may be taken to create the synthetic quadrature channel. The derivative may be a discrete derivative (i.e., a running difference). In an embodiment, the derivative may be formed by (1) setting a first lagging point to zero, (2) for the first through the nth point, subtracting the n−1 point from the n+1 point, and (3) dividing by 2. FIG. 4(d) shows a synthetic quadrature channel formed by the above derivative function.

In step 314.4, a variance, Var2, of the synthetic quadrature channel data may be calculated from the main pulse (i.e., PEAK) through to the end of the data for later scaling purposes if needed. The variance may correspond to the distribution of the data samples from the PEAK.

In step 314.5, if the synthetic quadrature channel data displays unequal characteristics, the synthetic quadrature channel data may be normalized. If the quadrature channel (say a cosine function) is then formed by taking the derivative of the in-phase channel (say a sine function) absent a scaling function, the synthetic quadrature channel may include a biasing error. Thus, a scaling function may equalize the in-phase and quadrature values in power.

In an embodiment, the scaling factor may be based on the previously calculated Var1 and Var2 values. For example, the scaling factor may equal

$$\sqrt{\frac{Var 1}{Var 2}} .$$

For normalization, the synthetic quadrature channel may be multiplied by the scaling factor. FIG. 4(e) shows a normalized synthetic quadrature channel.

In step 316, envelope detection may be performed using the in-phase and synthetic quadrature channel data. In an embodiment, envelop detection may be performed by taking the root mean squared (RMS) of the two data sets (i.e., \sqrt{ΔSignal_{I}²+ΔSignal_{Q}²}). The envelope detection may remove the zero crossings as shown in FIG. 4(f), which may make the data more amenable for further data interpretation processing such as threshold comparisons and widowed correlations.

In step 318, if the data displays noisy characteristics, the data may be low pass filtered. In an embodiment, the low pass filter may remove leading and falling edge noise; hence, the peaks may be easier to detect. As a result, threshold comparison without hysteresis may be more accurately applied later. In an embodiment, a boxcar filter in the form of an N point filter (ex. N=27) may be used. Other weighted feedback filters may also be used. FIG. 4(g) shows low pass filtered results from the above-described filtering process.

In step 320, the results may be interpreted. The results may indicate the presence or absence of a medical condition and may further estimate the medical condition's characteristics. For example, in a pneumothorax monitoring scenario, the magnitude may indicate the size of the air gap because the magnitude may be proportional to the integrated volume of the air gap. Further, the time may indicate the depth/location of the air gap because the time of a deviation (e.g., sample number) may be proportional to the depth of air gap. For example, the results may be compared to stored profiles of known medical conditions and if the results match a certain profile, it may indicate the presence of the corresponding medical condition. In an embodiment, the results may be translated into a graphical display element. The graphical display element may convey the changes in the patient's condition as measured by the target scan(s). For example, the graphical display element may be an icon that changes size and/or color based on the results. The display may be provided on the detector device, the host system or both.

Systems of the present invention may be used for several medical applications, particularly for monitoring and diagnostic purposes. For example, systems of the present invention can be used to monitor and collect data based on tissue permittivity changes. Conditions that result in tissue permittivity changes include pneumothoraces, perforated bowels and hematomas.

A pneumothorax may be induced by several causes including the placement of an intravascular device such as a central venous access device including, for example, central venous catheters (CVCs) and peripherally inserted central catheters (PICCs). Systems of the present invention can be used to detect the presence of a pneumothorax and additionally may be used to detect the size and/or location of the pneumothorax. The systems may be employed in the clinical setting in order to diagnose possible procedure complications such as the practitioner accidentally contacting the pleural lining of the lung while attempting to access the entry vessel. Traditionally, methods such as x-ray or computer tomography (CT) scans have been used to ensure the absence of pneumothoraces. However, using systems of the present invention are less expensive, faster, can be performed while the patient is undergoing the procedure and do not subject the patient or hospital staff to high doses of radiation.

A perforated bowel is a complication that may occur during a colonoscopy and polypectomy. Early on, bowel sounds may be preserved but typically are absent when the presentation of the perforation is delayed and peritonitis becomes established. When an immediate perforation is suspected, plain and upright x-rays of the abdomen are generally performed to confirm whether a perforation has occurred. In the case of delayed presentation, the patient generally exhibits high fever and leukocytosis. If plain films such as x-rays fail to demonstrate free air in the peritoneum, an abdominal CT scan is generally performed because of its higher sensitivity for this finding. In any event, prompt diagnosis and directed management are required to enhance a favorable outcome. The systems of the present invention may be used inter-operatively to detect the presence of air being built up in the abdominal cavity and to warn the practitioner immediately prior to the patient becoming symptomatic.

The systems of the present invention can also be used to detect and monitor pericardial effusion, pleural effusion or other pooling of fluids in or around tissues; stomach content changes or distention; changes in bone growth; respiratory function during anesthesia delivery; tumor progression; hemorrhages or aneurysms; and onset of kidney or gallstones.

The systems of the present invention can be integrated with other medical devices and kits to provide integrated diagnostic or monitoring systems. Exemplary medical devices include implantable or insertable medical devices including intravascular medical devices.

The MIR data collected from systems of the present invention may be used as a feedback tool for an algorithm that may be used to trigger or refine the response from a therapy delivering medical device. For example, a detector and antenna of the present invention can be used with a device that intakes the MIR data collected by the detector device and subsequently uses the data to control therapy delivery in a medical device.

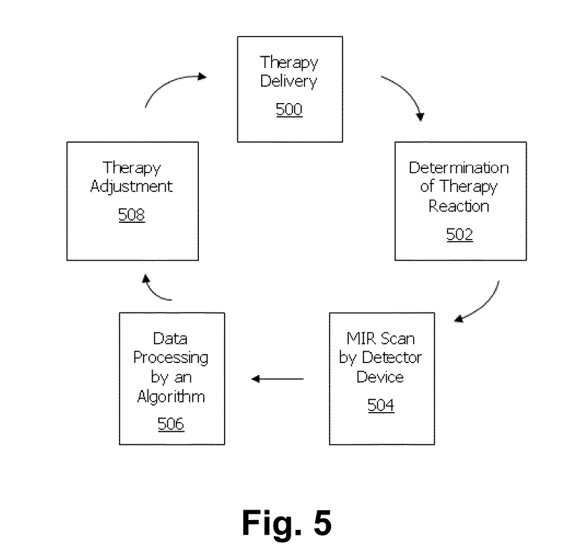

A flow diagram illustrating the steps of such a method is depicted in FIG. 5. As illustrated, FIG. 5 shows a feedback loop using systems of the present invention to control therapy delivery in a medical device according to an embodiment of the present invention. At step 500, therapy delivery is initiated. At steps 502 and 504, the system determines the reaction of the therapy on a patient and performs an MIR scan by a detector device (described above with respect to FIGS. 2 and 3). Data generated by the MIR scan is then processed by a processing device in step 506. In response to the results of the data processing in step 506, the therapy delivery is adjusted to refine the response from the therapy delivering medical device.

In more detail, embodiments of the present invention allow MIR data from a system of the present invention to be incorporated into the therapy delivery algorithm of another medical device to create an integrated system. For example, in one embodiment, the therapy delivery device is a cardiac pacemaker or left ventricular assist device (LVAD). A pacemaker delivers electrical signals that time the contraction of the heart. One of the factors that may affect a patient with a pacemaker and congestive heart failure is pericardial effusion, or excessive fluid around the heart. The MIR data collected by a detection device of the present invention may be taken in the intensive care unit (ICU) in the first days after an antenna device of the present invention is affixed to the patient and feed information via the detection device on the severity of the pericardial effusion back to the pacemaker. For example, initial MIR data collected may be stored as reference scan data and subsequent target scan(s) may monitor the effusion. This allows a system of the present invention to recognize whether the effusion is getting better or worsening, and possibly control the electrical pulses delivered by the pacemaker based on the severity of the effusion as monitored by MIR target scan(s).

Systems of the present invention may be used with other therapy delivery medical devices to provide a feedback mechanism. For example, MIR scans from systems of the present invention may be used to detect stomach content changes or distention. MIR data provided by the systems of the present invention may be used to control therapy delivery information to a stimulation device or lap band for obesity or other eating disorders. The systems of the present invention may also be used to detect changes in bone growth. MIR data from systems of the present invention can be used to control therapy delivery information to a bone growth stimulator. Data generated by the systems also can be used to control therapy delivery information to other electrical stimulation devices such as, for example, neural stimulations (both brain, spinal, and nerve), muscle stimulators, and skin stimulators. The systems of the present invention may also be used to collect lung data during anesthesia delivery to control anesthesia setting and breathing. MIR scans from exemplary systems also may be used to provide respiratory data to improve a pacemaker/defibrillator algorithm. MIR scans from exemplary systems may also be used to track tumor progression to control implanted cancer drug delivery devices.

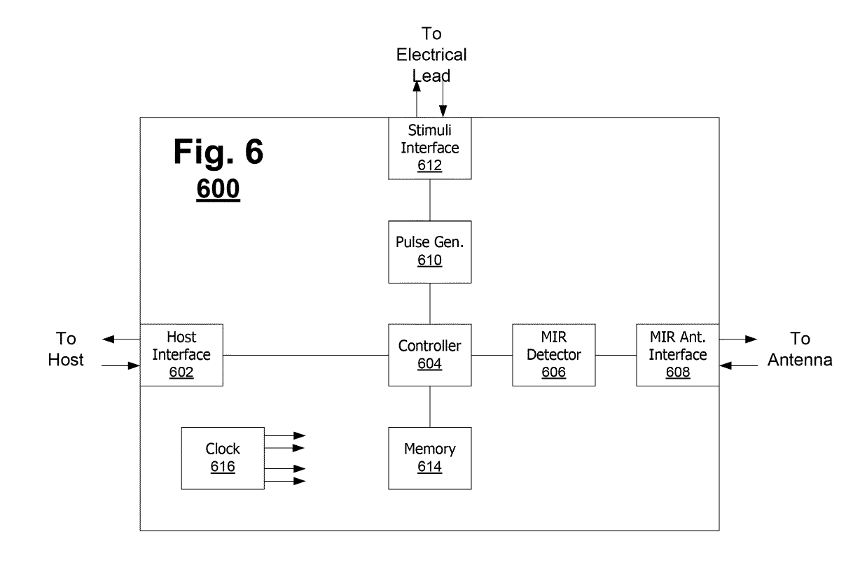

FIG. 6 is a simplified block diagram of an integrated MIR/stimulation system. The system 600 may include a host interface 602, a controller 604, an MIR detector device 606, an MIR antenna interface 608, a pulse generator 610, a stimuli interface 612, a memory 614, and a clock 616.

The host interface 602 may couple the system 600 to a remote host system such as a laptop, notebook, tablet computer, desktop computer or the like. In an embodiment, the interface 600 may be a USB port. In another embodiment, the host interface 602 may facilitate wireless communication with the host system such as by long range communication (e.g. cellular), short range communication (e.g., WIFI, Bluetooth) or a combination thereof.

The controller 604 may be a microcontroller or a microprocessor. The controller 116 may execute the instructions stored in the memory 614 and may control the operations of the system 600.

The MIR detector device 606 may generate, transmit/receive, and process MIR scans as disclosed in various embodiments herein.

The MIR antenna interface 608 may couple system 600 to a MIR antenna device. In an embodiment, the MIR antenna interface 608 may support connection to a coaxial cable. In another embodiment, the MIR antenna interface 608 may be provided as a wireless communication interface for networks such as WIFI, Bluetooth or the like.

The stimuli interface 612 may provide an output device for transferring the electric stimuli generated by the pulse generator 608 to a specified target site. The stimuli interface 612 may be contacts for an electrical lead.

The memory 614 may be provided as a volatile memory, a non-volatile memory, or a combination thereof. The memory 614 may store program instructions, scan data generated by the system 600 and any pattern data as needed by the system 600.

The clock 616 may provide timing signals for the various system 600 components such as the controller 604, the MIR detector device 606, the pulse generator 610, the memory 614, etc.

The pulse generator 610 may generate electrical pulses, which may be a form of medical therapy stimuli. The pulse generator 610 may generate the electrical pulses based on MIR scan data processed by the MIR detector device 606 as directed by the controller 604. In an embodiment, the pulse generator 610 may generate electric pulse stimuli (e.g., therapy) in response to target scan information from the MIR detector device 606 relating to positioning of an electric lead. For example, improper electric lead positioning may lead to the therapy electric pulse stimuli to be transmitted to unintended target sites. The MIR scans may detect whether the intended target site or unintended target site is receiving the electric pulse stimuli because the target sites may have different associated impedances or other characteristics and the electric pulse stimuli may change the composition of the target sites. The MIR scans may detect the change in the target site compositions and, consequently, the integrated system may determine proper or improper electrical lead positioning. Therefore, the integrated system of the present invention may improve therapy delivery mechanisms to provide for optimal therapeutic benefit. In another embodiment, the pulse generator 610 may generate electrical stimulations to a target site in response to target scan information from the MIR detector device 608 that indicate a fluid pooling or other abnormality.

Systems of the present invention can also be incorporated into wearable or home warning systems for acute medical attention. For example, systems of the present invention can be used as diagnostic tools for medical conditions that require continuous monitoring and alert both in and out of a clinical setting. In an exemplary embodiment, a detector device of the present invention collects MIR data and uses the data to alert a patient or practitioner of the need for acute medical attention outside of a clinic or hospital setting.

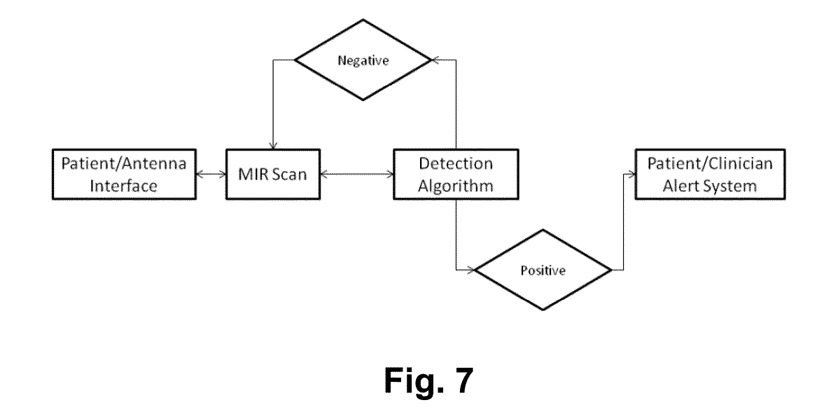

FIG. 7 is a flowchart of an MIR scan from a system of the present invention to detect and alert the patient or practitioner of the need for acute medical intervention. The system may perform MIRs scan using a detector device and antenna as described above with respect to embodiments of the present invention. The system may compare a reference scan and a target scan to determine whether medical conditions exist. If medical conditions do not exist (“negative”), the system may continue the MIR scanning process. If the system detects a medical condition (“positive”), the system may trigger a patient/clinician alert system to alert an attending physician or nurse of the medical condition.

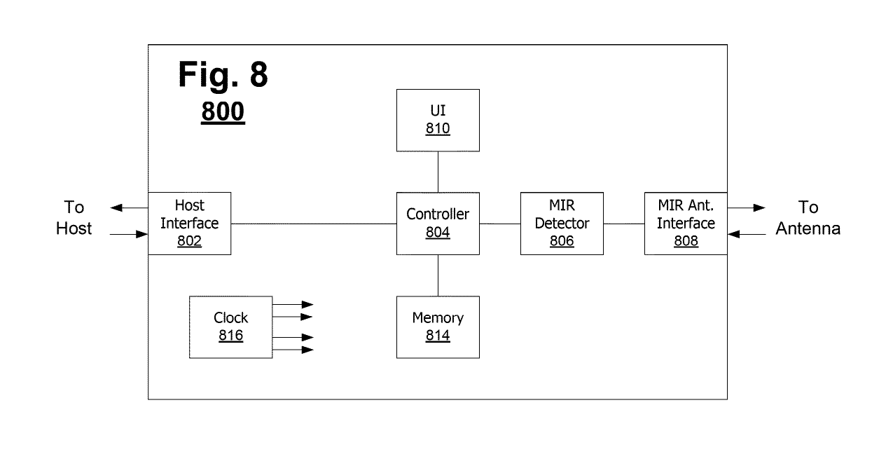

FIG. 8 is a block diagram of an integrated system 800 that may allow for certain patient conditions to be continuously monitored so that acute medical intervention may be given quickly if an MIR scan detects an anomaly according to an embodiment of the present invention. The integrated system 800 may include a host interface 802, a controller 804, an MIR detector device 806, and MIR antenna interface 808, a memory 814, and a clock 816 (all of which are substantially similar to the corresponding components in system 600 of FIG. 6). The system 800 may also include a user interface 810.

In this embodiment, the MIR detector device 806 has the capability to communicate a scan anomaly with the patient or with appropriate clinical staff or emergency services. Communication with the patient may include simple signaling such as LED lights or sounds (e.g., user interface (UI) 810). Other alert mechanisms include WIFI, RF, or cell connection that would notify the appropriate personnel of the patient's location and condition.

Such alert systems may be used for sending patients home rather than keeping them for long observational stays in a hospital or in a clinical setting where patients may require continuous monitoring.

Non-limiting examples of how systems of the present invention can be integrated into acute medical intervention monitoring systems include using an MIR scan obtained from systems of the present invention to monitor pericardial effusion caused by endocarditis or congestive heart failure, for example. MIR scans obtained from systems of the present invention may also be used to detect hemorrhages or aneurysms, early onset of kidney or gallstones, and lung or breathing abnormalities associated with pleural effusion.

Systems of the present invention can also be used with cardiac pacemakers and other electrical stimulation devices. Pacemakers include a lead(s) that is inserted into the chambers of the heart. The lead has an electrode(s) attaches to its end that deliver electrical charge to the heart to regulate heartbeat. The electrodes are positioned on the areas of the heart that require stimulation. The leads are then attached to an implantable pulse generator that is usually implanted under the skin of the patient's chest.

Patients undergoing surgical pacemaking implantation usually stay in the hospital overnight to undergo monitoring of vital signs, pacing efficacy, and to confirm that no pneumothorax occurs. Lead location is confirmed during the procedure using fluoroscopy. Standard practice is to perform a routine chest X-ray to confirm the absence of a pneumothorax. The systems of the present invention may be used inter-operatively to monitor the patient to eliminate the necessity of an X-ray for pneumothorax detection.

There currently exist systems that can continuously monitor patients for vital signs and smart pacemakers that continually assess pacing quality. However, no system exists to continuously monitor a patient for a pneumothorax. MIR data collected from systems of the present invention can be integrated into a device that may be worn by the patient allowing them to be sent home immediately after the surgical implantation of the pacemaker. The systems of the present invention can also be integrated into current monitors that are used for assessing vital signs and pacemaker functionality.

Systems of the present invention can also be utilized in conjunction with catheter tip locating systems to both verify the placement of a catheter such as a CVC and ports placed under the skin and to detect a pneumothorax. Such an integrated system is described in more detail in U.S. Provisional Application No. 61/566,844 filed on Dec. 5, 2011 which is incorporated by reference herein.

The foregoing description has been set forth merely to illustrate the invention and is not intended as being limiting. Each of the disclosed aspects and embodiments of the present invention may be considered individually or in combination with other aspects, embodiments, and variations of the invention. Further, while certain features of embodiments of the present invention may be shown in only certain figures, such features can be incorporated into other embodiments shown in other figures while remaining within the scope of the present invention. In addition, unless otherwise specified, none of the steps of the methods of the present invention are confined to any particular order of performance. Modifications of the disclosed embodiments incorporating the spirit and substance of the invention may occur to persons skilled in the art and such modifications are within the scope of the present invention. Furthermore, all references cited herein are incorporated by reference in their entirety.

We claim:

1. A detector device comprising:
transmitting circuitry to generate micropower impulse radar signals and transmit a first signal to perform a reference scan and a second signal to perform a target scan;
receiving circuitry to receive reflection data in response the reference scan and the target scan; and
a processor to compare the reflection data of the reference scan and the reflection data of the target scan to determine if a medical condition is present.

2. The device of claim 1, wherein:
the transmitting circuitry transmits the first and second signals to an antenna that transmits electromagnetic waves in response to the signals; and
the receiving circuitry receives reflection data associated with the first and second signals from the antenna.

3. The device of claim 1, wherein the detector device continuously monitors for medical conditions by sending an initial reference scan and subsequent target scans.

4. The device of claim 1, wherein the medical condition is a pneumothorax.

5. The device of claim 1, wherein the reference scan is performed when the medical condition is not present.

6. The device of claim 1, wherein the processor uses envelope detection with a synthetic quadrature channel to analyze the reflection data of the reference scan and the target scan.

7. The device of claim 1, wherein the detector device is integrated with a therapy delivering medical device to refine a response from the therapy delivering medical device.

8. The device of claim 1, wherein the detector device is integrated with a patient alert system that provides an alert in response to a determination by the detector device that the medical condition is present.

9. The device of claim 1, wherein the detector device is integrated with a catheter tip location system to verify a placement of a catheter and determine if the medical condition is present as a result of the catheter placement.

10. A diagnostic system, comprising:
a transmitter to transmit stimulus signals to tissue subject to study,
a receiver to receive reflected signals returned from the tissue in response to the stimulus signal, and
a processor to compare characteristics of a first reflected signal received in response to a first stimulus signal to characteristics of a second reflected signal received in response to a second stimulus signal and, based on the comparison, to indicate an error condition.

11. The diagnostic system of claim 10, wherein the processor initiates transmission of stimulus signals at timed intervals and compares characteristics of the first reflected signal to characteristics of a plurality of reflected signals received in response to the timed stimulus signals.

12. The diagnostic system of claim 10, wherein the first stimulus signal is transmitted prior to commencement of a medical procedure and the second stimulus signal is transmitted after commencement of the medical procedure.

13. A system comprising:
a detector device to perform a reference scan and a target scan by generating first and second micropower impulse radar pulses and sending the pulses to an antenna;
an antenna to:
receive the pulses from the detector and transmit signals in response to each of the pulses; and
receive reflection signals corresponding to each of the transmitted signals, and
a processor to compare the reflection signals corresponding to the reference scan and the target scan to determine if a medical condition is present.

14. The system of claim 13, wherein the system continuously monitors for medical conditions by performing an initial reference scan and subsequent target scans.

15. The system of claim 13, wherein the medical condition is a pneumothorax.

16. The system of claim 13, wherein the reference scan is performed when the medical condition is not present.

17. The system of claim 13, wherein the processor uses envelope detection with a synthetic quadrature channel to analyze the signals.

18. The system of claim 13, wherein the system is integrated with a therapy delivering medical device to refine a response from the therapy delivering medical device.

19. The system of claim 13, wherein the system is integrated with a patient alert system that provides an alert in response to a determination by the processor that the medical condition is present.

20. A method comprising:
performing a reference scan by:
transmitting a first stimulus signal into tissue subject to scan; and
receiving reflected signals in response to the first stimulus signal; and
at some time after the reference scan, performing a target scan by:
transmitting a second stimulus signal into tissue subject to scan, the first and second stimulus signals having common characteristics; and
receiving reflected signals in response to the second stimulus signal; and
comparing characteristics of the reflected signals of the reference scan to characteristics of the reflected signals of the target and, based on the comparison, generating a notification indicating that a medical condition is present.

21. The method of claim 20, further comprising:
performing subsequent target scans at timed intervals,
comparing reflected signals of the subsequent scans to the reflected signals of the reference scan, and
based on the comparisons of the subsequent target scans, generating a notification indicating that a medical condition is present.

22. The method of claim 20, wherein the medical condition is a pneumothorax.

23. The method of claim 20, wherein the reference scan is performed when the medical condition is not present.

24. The method of claim 20, further comprising using envelope detection with a synthetic quadrature channel to analyze the reflection signals.

25. The method of claim 20, further comprising refining a response from a therapy delivering medical device based on the comparison.

26. The method of claim 20, further comprising providing an alert if the medical condition is present.
